(19) United States
(12) Patent Application Publication
(10) Pub. No.: US 2006/0292101 A1
(43) Pub. Date: Dec. 28, 2006

(54) In-eye method of cleaning and/or disinfecting silicone hydrogel contact lenses

(76) Inventors: Roya Borazjani, Forth Worth, TX (US); Joseph C. Salamone, Boca Raton, FL (US); Vicki Barniak, Fairport, NY (US); Catherine Scheuer, West Henrietta, NY (US)

Correspondence Address: Bausch & Lomb Incorporated, One Bausch & Lomb Place, Rochester, NY, 14604-2701, US

(21) Appl. No.: 11/475,278

(22) Filed: Jun. 27, 2006

Related U.S. Application Data
(60) Provisional application No. 60/694,791, filed on Jun. 28, 2005.

(51) Int. Cl. A61K 8/73; A61K 8/33

(52) U.S. Cl. 424/70.13; 424/70.31; 510/110

(57) ABSTRACT

A method of treating a contact lens, and in some instances, a silicone hydrogel contact lens, in the eye of a wearer comprising i) applying to said contact lens and/or eye an ophthalmically compatible contact lens cleaning and/or hydrating and/or disinfecting solution; and ii) rubbing the eyelid associated with said eye over the contact lens for at least about one second.

### CROSS REFERENCE

This application claims the benefit of Provisional Patent Application No. 60/694,791 filed Jun. 28, 2005 and is incorporated herein by reference.

### FIELD

The present invention relates generally to a method of applying a cleaning and/or disinfecting solution in the form of eyedrops to a contact lens while it is worn in the eye, followed by rubbing of the eyelid, which method improves the effectiveness of the solution. The method is especially useful for improving the effectiveness of a solution used with a silicone hydrogel contact lens, especially for preventing the deposition of lipid and lipid-like substances on the lens.

### BACKGROUND

Maintaining the cornea free from harmful bacteria, especially during continuous wearing of contact lenses, is of particular importance to ocular health and prevention of contact lens associated complications. The corneal surface is considered a sterile environment, except for transient microorganisms that are mostly skin and lid associated, such as Staphylococcus Spp.

The contact lens can act as a vector that transports microorganisms from the contaminated environment to the eye, as well as a possible substratum for proliferation of bacteria. Contact lenses can provide a surface on which bacteria can proliferate and produce threshold numbers for corneal invasion and/or antigenic mass, e.g., biofilm. A major concern with contact lenses, including high Dk lenses, particularly with individuals having low tear film flow, is that the lenses impede normal tear flow. The end result may be the build up of deposits on the lens at particular sites. The deposits may serve as foci for the attachment of bacteria and/or development of a primary adhesion layer. Currently available high Dk extended wear silicone lenses typically contain from about 24% to about 48 wt % water and provide more compliant surfaces for attachment of select pseudomonads than non-silicone type lenses of similar water content.

Contact lenses in wide use today fall into two categories. First, there are the hard or rigid corneal type lenses that are formed from materials prepared by the polymerization of acrylic esters, such as poly(methyl methacrylate) (PMMA). Secondly, there are the gel, hydrogel or soft type of lenses made by polymerizing such monomers as 2-hydroxyethyl methacrylate (HEMA). Of particular interest is a class of high Dk soft lenses made from polymers comprising siloxy-containing monomers and/or macromonomers.

Contact lenses made from siloxy-containing materials have been investigated for a number of years. Such materials can generally be subdivided into two major classes, namely hydrogels and non-hydrogels. Non-hydrogels do not absorb appreciable amounts of water, whereas hydrogels can absorb and retain water in an equilibrium state. Regardless of their water content, both non-hydrogel and silicone hydrogel contact lenses tend to have relatively hydrophobic, non-wettable surfaces.

Those skilled in the art have long recognized the need for modifying the surface of such silicone hydrogel contact lenses so that they are compatible with the eye. It is known that increased hydrophilicity of the contact lens surface improves the wettability of the contact lenses. This in turn is associated with improved wear comfort of contact lenses. Additionally, the surface of the lens can affect the lens's susceptibility to deposition, particularly protein and lipid deposition from the tear fluid during lens wear. Accumulated deposition can cause eye discomfort or even inflammation. In the case of extended wear lenses, the surface is especially important since extended wear lenses must be designed for high standards of comfort over an extended period of time, without requiring daily removal of the lens before sleep. Thus, the regimen for the use of extended wear lenses would not provide a period of time on a daily basis for the eye to rest or recover from any discomfort or other possible adverse effects due to lens wear during the day.

The patent literature has disclosed various surface treatments for rendering the surface of silicone hydrogel lenses more hydrophilic and more wettable, including changing the chemistry of the surface layer, coating the surface, and compounding the polymer with additives that subsequently diffuse to the surface. Among chemical surface modification techniques are non-polymeric plasma treatments and corona treatments. The surface of a contact lens can also be modified, at least temporarily and to various degrees, by treatment with contact-lens care solutions.

Solutions that wet the lenses before insertion in the eye are required for both the hard and soft types of contact lenses, at least for non-disposable lens or lenses that are reused at least once after being worn. Surfactant cleaning agents in daily lens care solutions are useful for the removal of lens lipids. Also, the use of enzymes or equivalent protein removing agents has been conventional. With the advent of extended wear lenses, however, in which lenses are worn overnight and even continuously over a plurality of days, the conventional lens care solutions no longer have the opportunity to remove depositions that have accumulated over the day with daily cleaning solutions. Also, because of the hydrophobicity of silicone hydrogel materials, they are especially susceptible to the deposition of lipid or lipid-like materials.

It would therefore be desirable to have a solution that could be applied in an efficient manner to the eye in order to accomplish cleaning, disinfecting, and/or preventing the deposition of lipids or other materials, until such time as the lens is removed from the eye and cleaned or disposed.

It does not necessarily follow that cleaning agents used in cleaning solutions in which the contact lenses are immersed for several hours or more would be effective when applied in the form of eyedrops. In particular, cleaning agents that are designed to prevent the deposition of lipids on the lens must have an extended effect in conjunction with the lens. At the same time, cleaning agents must be selected that are very safe and comfortable, especially as they would be expected to associate with the lens surfaces. Eye irritation must be avoided.

Ophthalmic solutions for rewetting, lubricating, and/or enhancing wearer comfort by application to the eye or a contact lens while being worn in the eye, are known. Rewetting solutions usually contain a wetting agent in combination with a biocide or preservative, a viscosity builder, and salts that adjust the tonicity of the solutions to make them compatible with the osmolality of tear fluids. Hypotonic to isotonic solutions for improving the comfort of wearing soft contact lenses are known. Such solutions typically may also contain lubricants or demulcents, surfactants, and/or buffers.

For example, U.S. Pat. No. 4,529,535 to Sherman discloses a rewetting solution that is particularly useful for silicone hydrogel contact lenses, including extended wear lenses. One embodiment includes the combination of hydroxyethylcellulose, poly(vinyl alcohol), and poly(N-vinylpyrrolidone). U.S. Pat. No. 4,786,436 to Ogunbiyi discloses a wetting solution comprising collagen and other demulcents such as hydroxylethylcellulose, methylcellulose, carboxymethylcellulose, hydroxypropylmethylcellulose, hydroxylpropylcellulose and the like.

U.S. Pat. No. 4,748,189 to Su et al. discloses an ophthalmic solution for improving the exchange of fluid in the area outside a hydrogel contact lens in the area underneath the hydrogel contact lens, in order to permit tear exchange to occur, thereby preventing the accumulation of waste matter and debris under the lens. The solution contains a hydrogel flattening agent, for example urea, glycerin, propylene glycol, sorbitol or an aminoethanol. Surfactants that are useful in the solution include poloxamer and tyloxapol. Suitable lubricants include alcohol hydroxylethylcellulose, poly(vinyl alcohol), and poly(N-vinylpyrrolidone).

U.S. Pat. No. 5,209,865 to Winterton et al. discloses a conditioning solution for contact lenses that comprises a combination of a poloxamine and a poloxamer surfactant each having an HLB (hydrophilic-lipophilic balance) of seven or below. The solution according to the invention forms a uniform hydrophilic film on a lens surface for which proteins have very little affinity. As such, a contact lens contacted by the solution is said to have a coating that provides a prophylactic effect to the lens.

U.S. Pat. Nos. 4,436,730 and 4,401,327 to Ellis et al. disclose the use of cationic cellulosic derivatives in contact-lens treating solutions, including the combination of a cationic cellulose such as Polymer JR-30M and an ethoxylated glucose such as glucam. In column 4, lines 42-57, Ellis et al. state that the combination of a cationic cellulose material with a PEO [(poly(ethylene oxide)] component such as glucam is particularly advantageous for the reason that the cationic component complexes with the PEO component and the complex more strongly adsorbs on the lens surface. The cationic cellulose polymer and entangled PEO are believed to reach into the aqueous phase to provide cushioning and protein resistance. The invention covered by these patents, however, has been used in products for RGP (rigid-gas-permeable) silicone-containing lenses as compared to soft lenses.

U.S. Pat. No. 6,274,133 to Hu et al. discloses a method for treating silicone hydrogel extended wear contact lenses in the eyes, utilizing an ophthalmic solution containing a cationic cellulosic polymer that binds to the lens and prevents accumulation of lipids, proteins and other products. The solution is applied in the form of drops to a contact lens in the eye.

U.S. Pat. No. 6,872,695 to Groemminger et al. teaches an in-eye method for cleaning contact lenses, e.g., silicone hydrogel lenses, with compositions comprising water-containing beads that are easily flushed from the ocular environment by normal tear flow. The method can comprise the sequential steps of blinking and then rinsing the eye and the contact lens with rinsing solution to remove the cleaning solution from the eye.

It would be desirable to provide a method of administering an eye drop or a multipurpose solution that can condition and clean a silicone hydrogel contact lens while in the eye, providing additional disinfection properties on the lens during wear which prevent the overgrowth of transient microorganisms (bioburden) known as normal flora, on the lens surface.

### SUMMARY

In one aspect, the present invention relates to a method of treating a contact lens, particularly a silicone hydrogel contact lens, in the eye of a wearer comprising i) applying to said contact lens and/or eye an ophthalmically compatible contact lens solution, e.g., cleaning and/or hydrating and/or disinfecting solution comprising hypotonic to isotonic buffer, wetting agent, conditioning agent, sequestering agent, preservative, and/or biocidal agent; and ii) rubbing the eyelid associated with said eye over the contact lens for at least about one second, say, between about two seconds and 10 seconds, e.g., at least about 5 seconds.

In one embodiment, the ophthalmic solution further comprises at least one additional component selected from surfactant and wetting agent.

In another embodiment, the method further comprises the sequential steps of blinking and then rinsing the eye and the contact lens with an ophthalmic rinsing solution to dilute or substantially remove the contact lens cleaning solution from the eye.

In still another embodiment, the contact lens is an extended wear lens worn within the range of about 7 days to about 30 days and the method improves resistance to lipid deposition as measured by lipid solubility method as measured by a lipid solubility method using spectrophotometrically determined optical density.

In yet another embodiment, the solution is a sterile aqueous solution comprising: (a) 0.01 to 1.0 percent by weight of a cationic cellulosic polymer; (b) at least one tonicity agent which is present in an amount of 0.01 to 10.0% by weight; and (c) an effective amount of a buffering agent to maintain the pH from about 6 to about 8. The solution can further comprise 0.1 to 2.0 percent by weight of one or more surfactants, e.g., a poly(oxyethylene)-poly(oxypropylene)-poly(oxyethylene) nonionic surfactant.

In another embodiment, the solution further comprises a wetting agent. The wetting agent can be selected from the group consisting of glycerin, propylene glycol, monosaccharide, disaccharide, poly(ethylene glycol), ethoxylated glucose, and combinations thereof.

In yet another embodiment, the solution further comprises a polymeric nonionic demulcent. The demulcent can be selected from hydroxyethylcellulose, hydroxypropylcellulose, guar, hydroxyethylguar, hydroxypropylmethylguar, carboxymethylcellulose, povidone, and poly(vinyl alcohol).

In still another embodiment, the solution further comprises an effective amount of a sequestering agent. The sequestering agent can be selected from ethylenediaminetetraacetic acid and 2-hydroxyethyl-1,1-bis(phosphonic acid), and derivatives thereof.

In yet another embodiment, the solution is applied by an eye-drop dispenser and is stored in a container capable of holding between about 1 and about 30 ml of the solution.

In yet still another embodiment, the solution is employed to clean the lens.

In another embodiment, the solution is employed to clean and/or prophylactically clean lipid and/or protein deposits on the lens.

In still another embodiment, the method results in a reduction of bacteria on the surface of said lens which contacts the eye ranging from about 20% to about 90%, preferably from about 40% to about 60% as measured before applying the solution and immediately after said rubbing by viable count method, e.g., to determine the number of viable colonies on the lens (CFU/lens).

In yet another embodiment, steps i) and ii) are repeated at least once.

In still another embodiment, the rubbing is carried out by rubbing a fingertip over the closed eyelid.

The present invention improves the bioburden reduction characteristic Bioburden refers to the number of alive transient microorganisms on the surface of the cornea, referred to as normal flora which can attach to the surface of the contact lens specially during extended wear. The present invention helps remove the majority of attached microorganisms to the surface of contact lenses during wear. The present invention also improves tear-film-deposit removal properties of the cleaning and/or disinfecting solution, particularly for extended wear periods, e.g., from about 1 week to about 30 days. The method of the present invention is useful in preventing or reducing attachment and therefore overgrowth of harmful bacteria, including both Gram-positive and Gram-negative species, e.g., Pseudomonas aeruginosa, Serratia marcescens and Staphylococcus aureus, as well as harmful molds, e.g., Acanthamoebae, on the lens surfaces during wearing. Moreover, the present method is especially useful inasmuch as it is gentle and non-toxic with respect to corneal epithelial cells.

### DETAILED DESCRIPTION

The present invention is directed to a method for cleaning and/or disinfecting a contact lens while it is worn in the eye and which is also useful for prophylactically cleaning the lens by preventing the deposition of lipids or other depositions on the lens. In particular, the present invention is useful with respect to extended wear lenses that are made from a silicone-hydrogel material. Lenses made from silicone-hydrogel or fluorosilicone hydrogel materials are more hydrophobic than other types of soft lenses and are, therefore, especially prone to lipid deposition which may present a problem during the extended use of such lenses.

Hydrogels in general are a well-known class of materials that comprise hydrated, crosslinked polymeric systems containing water in an equilibrium state. Silicone hydrogels generally have a water content greater than about 5 weight percent and more commonly between about 10 to about 80 weight percent. Such materials are usually prepared by polymerizing a mixture containing at least one siloxy-containing monomer (including macromonomers) and at least one hydrophilic monomer. Either the siloxy-containing monomer or macromonomer or the hydrophilic monomer can function as a crosslinking agent (a crosslinker being defined as a monomer having multiple polymerizable functionalities). Alternately, a separate crosslinker may be employed. Applicable siloxy-containing monomeric units for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

Another class of representative silicone-containing monomers includes siloxy-containing vinyl carbonate or vinyl carbamate monomers such as: 1,3-bis[4-vinyloxycarbonyloxy)but-1-yl]tetramethyldisiloxane; 3-(trimethylsilyl)propylvinyl carbonate; 3-(vinyloxycarbonylthio)propyl[tris(trimethylsiloxy)silane]; 3-[tris(trimethylsiloxy)silyl]propylvinyl carbamate; 3-[tris(trimethylsiloxy)-silyl] propyl allyl carbamate; 3-[tris(trimethylsiloxy)-silyl] propyl vinyl carbonate; t-butyldimethylsiloxyethyl vinyl carbonate; trimethylsilylethyl vinyl carbonate; and trimethylsilylmethyl vinyl carbonate.

Another class of silicon-containing monomers includes polyurethane-polysiloxane macromonomers (also sometimes referred to as prepolymers), which may have hard-soft-hard blocks like traditional urethane elastomers. They may be end-capped with a hydrophilic methacrylate monomer. Examples of such silicone urethanes are disclosed in a variety or publications, including Lai, Yu-Chin, “The Role of Bulky Polysiloxanylalkyl Methacrylates in Polyurethane-Polysiloxane Hydrogels,” Journal of Applied Polymer Science, Vol. 60,1193-1199 (1996). PCT Published Application No. WO 96/31792 discloses examples of such monomers, which disclosure is hereby incorporated by reference in its entirety.

A preferred silicone hydrogel material comprises (in bulk formula, that is, in the monomer mixture that is copolymerized) 5 to 50 percent, preferably 10 to 25, by weight of one or more silicone macromonomers, 5 to 75 percent, preferably 30 to 60 percent, by weight of one or more (poly)siloxanylalkyl (meth)acrylic monomers, and 10 to 50 percent, preferably 20 to 40 percent, by weight of a hydrophilic monomer, or combination of monomers, as a percentage of the hydrogel polymer material. In general, the silicone macromonomer is a poly(organosiloxane) capped with an unsaturated group at one or more ends of the molecule, typically two or more ends for copolymerization. In addition to the end groups in the above mentioned-patents, U.S. Pat. No. 4,153,641 to Deichert et al. discloses additional unsaturated groups, including acryloyloxy or methacryloyloxy.

Suitable hydrophilic monomers for use in silicone hydrogels include, for example, unsaturated carboxylic acids, such as methacrylic and acrylic acids; acrylic substituted alcohols, such as 2-hydroxyethyl methacrylate and 2-hydroxyethyl acrylate; vinyl lactams, such as N-vinylpyrrolidone (NVP); and acrylamides, such as methacrylamide and N,N-dimethylacrylamide, and the like. Still further examples are the hydrophilic vinyl carbonate or vinyl carbamate monomers disclosed in U.S. Pat. Nos. 5,070,215, and the hydrophilic oxazolone monomers disclosed in U.S. Pat. No. 4,910,277.

The lens care ophthalmic solutions useful in the present invention are those found to be particularly effective for use with silicone-hydrogel contact lenses as described above, which may be coated with materials or surface-modified to mask to some extent the hydrophobic nature of the core material. Examples of these materials include ReNu® MultiPlus, e.g., ReNu® with MoistureLoc™ Multi-Purpose Solution, available from Bausch & Lomb of Rochester, N.Y., USA. This solution contains boric acid, sodium chloride, sodium phosphate, hydroxyalkylphosphonate, poloxamine, poloxamer, polyquaternium-10, preserved with alexidine (0.00045%), and is covered by, inter alia, U.S. Pat. Nos. 4,820,352, 4,758,595, 4,836,986, 5,858,937, 6,274,133 and 6,309,658, all of which are incorporated herein by reference in their entirety.

Other lens-care ophthalmic solutions products suitable for use in the present invention include those characterized by OPTI-FREE® SupraClens® Daily Protein Remove, NO RUB™ OPTI-FREE® EXPRESS® Multi-Purpose Disinfecting Solution (MPDS) with ALDOX® Antimicrobial, available from Alcon of Fort Worth, Tex. USA, and AQuify® Multipurpose Solution, Ciba Vision of Duluth, Ga., USA.

A suitable cleaning/hydrating/disinfecting solution for use according to the present invention typically involves the combination of about 0.01 to 1.0 percent by weight of a cationic cellulosic polymer, at least one tonicity agent which is present in an amount of 0.01 to 10.0% by weight, and an effective amount of a buffering agent (which may also contribute to tonicity).In one embodiment, the solution also comprises a polymeric demulcent. It has been found that this particular combination of ingredients is both comfortable for use in the eye and effective in preventing the deposition of lipids. Without wishing to be bound by theory, the cationic cellulosic polymer is believed to complex with the lens surface. The cationic cellulosic polymer may further anchor other components such as any optional surfactant and/or polymeric demulcent on the surface and the entangled surfactant or demulcent may reach into the aqueous phase to provide further cushioning and deposit resistance. These separate functions, however, should not be viewed as clearcut and exclusive. Any surfactants may also loosen deposits on the lens; wherein removal is assisted by the natural cleaning action of blinking. The cellulosic polymer, by providing ionic charge, in particular, may inhibit the deposition of lipids because it renders the surface less hydrophobic.

Any suitable cationic cellulosic material may be used in the practice of this invention. Examples include cellulosic polymers containing N,N-dimethylaminoethyl groups (either protonated or quaternized) and cellulosic polymers containing N,N-dimethylamino-2-hydroxylpropyl groups (either protonated or quaternized). Cationic cellulosic polymers are commercially available or can be prepared by methods known in the art. As an example, the quaternary nitrogen-containing ethoxylated glucosides can be prepared by reacting hydroxyethyl cellulose with a trimethylammonium substituted epoxide. Various preferred cationic cellulosic polymers are commercially available water soluble polymers available under the CTFA (Cosmetic, Toiletry, and Fragrance Association) designation Polyquaternium-10, including the cationic cellulosic polymers available under the tradename UCARE™ Polymer from Amerchol Corp., Edison, N.J., USA). These polymers contain quaternized N,N-dimethylamino groups along the cellulosic polymer chain. Similarly, cationic polymers from other polysaccharides, such as cationic guar and gum derivatives, can be prepared.

The cationic cellulosic component may be employed in the compositions at about 0.001 to about 10 weight percent of the composition, preferably at about 0.02 to about 5 weight percent, with about 0.05 to about 2 weight percent being especially preferred. Suitable cationic cellulosic materials are described in U.S. Pat. No. 6,274,133, incorporated herein by reference.

The solutions suitably employed in the present invention preferably also contain a poly(oxyethylene)-poly(oxypropylene)-poly(oxyethylene) nonionic surfactant which, for example, can be selected from the group of commercially available surfactants having the name poloxamine or poloxamer, as adopted by The CTFA International Cosmetic Ingredient Dictionary. The poloxamine surfactants consist of a poly(oxypropylene)-poly(oxyethylene) adduct of ethylene diamine having a molecular weight from about 7,500 to about 27,000 wherein at least 40 weight percent of said adduct is poly(oxyethylene). These have been found to be particularly advantageous for use in conditioning contact lenses when used in amounts from about 0.01 to about 15 weight percent. Such surfactants are available from BASF Wyandotte Corp., Wyandotte, Mich., under the registered trademark “Tetronic”. The poloxamers are an analogous series of surfactants and are poly(oxyethylene), poly(oxypropylene)-poly(oxyethylene) block polymers available from BASF Wyandotte Corp., Parsippany, N.J. 07054 under the trademark “Pluronic”.

The HLB of a surfactant is known to be a factor in determining the emulsification characteristics of a nonionic surfactant. In general, surfactants with lower HLB values are more lipophilic, while surfactants with higher HLB values are more hydrophilic. The HLB values of various poloxamines and poloxamers are provided by BASF Wyandotte Corp., Wyandotte, Mich. Preferably, the HLB of the surfactant in the present invention is at least 8, preferably between 8 and 32, more preferably between 8 and 24, based on values reported by BASF.

Additional compatible surfactants that are known to be useful in contact wetting or rewetting solutions can be used in the solutions of this invention. The surfactant should be soluble in the lens care solution and non-irritating to eye tissues. Satisfactory non-ionic surfactants include polyethylene glycol esters of fatty acids, e.g. coconut, polysorbate, poly(oxyethylene) or poly(oxypropylene) ethers of higher alkanes (C₁₂-C₁₈). Examples of the preferred class include polysorbate 20 (available from ICI Americas Inc., Wilmington, Del. 19897 under the trademark Tween® 20), poly(oxyethylene) (23) lauryl ether (Brij® 35), poly(oxyethylene) (40) stearate (Myrj® 52), poly(oxyethylene) (25) propylene glycol stearate (Atlas® G 2612). Brij® 35, Myrj® 52 and Atlas® G 2612 are trademarks of, and are commercially available from, ICI Americas Inc., Wilmington, Del., USA.

Various other surfactants suitable for use in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

As indicated above, a polymeric demulcent can be advantageously included in the solution employed in the present invention. The demulcent provides wetting, moisturizing, and/or lubricating of contact lens in the eyes of wearers, resulting in their increased comfort. The polymeric demulcent can also act as a water-soluble viscosity builder. Because of their demulcent effect, viscosity builders have a tendency to enhance the lens wearer's comfort by means of adherence to the lens surface cushioning impact against the eye. Included among the water-soluble viscosity builders are the cellulose polymers like hydroxyethyl or hydroxypropylcellulose, hydroxypropylmethylcellulose, carboxymethylcellulose, povidone, poly(vinyl alcohol), and the like. Such viscosity builders or demulcents may be employed in a total amount ranging from about 0.01 to about 5.0 weight percent or less. Suitably, the viscosity of the final formulation is 10 cps to 50 cps. Comfort or wetting agents such as glycerin or propylene glycol can also be added and the former is particularly preferred, for example, in amounts ranging from 0.01 to 2.0 percent by weight of the solution. Other suitable wetting agents include mono or disaccharide, poly(ethylene glycol), ethoxylated glucose, and the like.

A preferred demulcent is povidone [(poly(N-vinylpyrrolidone) or PVP], which is a Category I demulcent in the OTC Ophthalmic Drug Products Monograph of the USFDA. Poly(N-vinylpyrrolidone) (PVP) is a linear homopolymer or copolymer comprising at least about 80%, preferably at least about 90% of repeat units derived from 1-vinyl-2-pyrrolidone monomers, the polymer more preferably comprising at least about 95% or essentially all of such repeat units, the remainder selected from polymerization-compatible monomers, preferably neutral monomers, such as alkenes or acrylates. Other synonyms for PVP include povidone, polyvidone, 1-vinyl-2-pyrrolidinone, and 1-ethenyl-2-pyrolionone (CAS registry number 9003-39-8). PVP has a weight average molecular weight of about 10,000 to 250,000, preferably 30,000 to 100,000. Such materials are sold by various companies, including ISP Technologies, Inc. under the trademark PLASDONE™ K-29/32, BASF under the trademark KOLLIDON™ for USP grade PVP, for example KOLLIDON™ K-30 or K-90 (BASF Corporation, NV Division, 3000 Continental, Mount Olive, N.J. 07628-1234, USA). In the present compositions, the PVP is suitably present in an amount 0.01 to 10.0% by weight, preferably of between 0.05 to 5.0 percent by weight.

The cleaning/hydrating/disinfecting composition suited to use in the present composition can contain a disinfecting amount of a preservative or an antimicrobial agent. Particularly preferred preservatives include alexidine, polyhexamethylene biguanide, and sorbic acid, with alexidine being the most preferred preservative in a concentration range of 1 ppm to 10 ppm. Antimicrobial agents are defined as organic chemicals which derive their antimicrobial activity through a chemical or physiochemical interaction with the microbial organisms. For example, biguanides include the free bases or salts of alexidine, chlorhexidine, hexamethylene biguanides and their polymers, and combinations of the foregoing. The salts of alexidine and chlorhexidine can be either organic or inorganic and are typically gluconates, nitrates, acetates, phosphates, sulfates, halides and the like. Other preferred antimicrobial agents are the polymeric quaternary ammonium salts used in ophthalmic applications and the biguanides, for example, the hexamethylene biguanides (commercially available from Zeneca, Wilmington, Del. under the trademark Cosmocil™ CQ), their polymers and water-soluble salts. Generally, the hexamethylene biguanide polymers, also referred to as polyaminopropyl biguanide (PAPB) or polyhexamethylene biguanide (PHMB), have molecular weights of up to about 100,000. Such compounds are known and are disclosed in U.S. Pat. No. 4,758,595 and British Patent 1,432,345, which patents are hereby incorporated herein by reference. The hydrochloride salt of polyhexamethylene biguanide is commercially available from Zeneca, Inc. under the trademark Cosmocil® CQ. This biguanide is often referred to as either “PHMB” or “PAPB,” as herein, usually by the latter acronym corresponding to polyaminopropyl biguanide.

In addition to the active ingredients described above, solutions employed in the present invention may contain buffers, stabilizers, tonicity agents and the like that aid in making ophthalmic compositions more comfortable to the user. The aqueous solutions of the present invention are typically adjusted with tonicity agents to either a hypotonic state, with an osmolarity between 220-280 mOsm/kg to and isotonic range of 280-320 mOsm/kg. The tonicity of the lens case solutions are adjusted with physiological saline, with glycerin or propylene glycol, with saccharides such as sorbitol, with buffers, or with combinations thereof. Correspondingly, excess salt or other tonicity agent may result in the formation of a hypertonic solution which will cause stinging and eye irritation. An osmolality of about 220 to 400 mOsm/kg is preferred, more preferably 225 to 280 mOsm/kg.

The pH of the present solutions should be maintained within the range of 5.0 to 8.0, more preferably about 6.0 to 8.0, most preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) aminoalcohols, Good buffers, and mixtures thereof. Borate buffers are preferred, particularly for enhancing the efficacy, with aminoalcohols and Good buffers being particularly preferred. Generally, buffers will be used in amounts ranging from about 0.05 to 2.5 percent by weight, and preferably, from 0.1 to 1.5 percent.

In addition to buffering agents, in some instances it may be desirable to include sequestering agents in the present solutions in order to bind metal ions that might otherwise react with the lens and/or protein deposits and collect on the lens, as well as deactivate biguanide preservatives. Ethylene-diaminetetraacetic acid (EDTA) and its salts (disodium) are preferred examples. They are usually added in amounts ranging from about 0.01 to about 0.2 weight percent.

As indicated above, the present invention is useful for cleaning a contact lens while it is worn in the eye. Thus, as mentioned above, the method of the present invention is especially advantageous with people who are prone to heavy lipid or other deposition or who wear lenses under an extended wear regime. Extended wear is defined as a lens that is worn overnight, during sleep, preferably capable of wear for a week, more preferably for two weeks, and most preferably for about one month.

The compositions used in the method of the present invention can be provided in a wide range of small volume containers, typically 1 to 30 ml in size, preferably 1 ml to 20 ml in size. Such containers can be made from HDPE (high density polyethylene), LDPE (low density polyethylene), polypropylene, poly(ethylene terepthalate) and the like. Flexible bottles having conventional eye-drop dispensing tops are especially suitable for use with the present invention.

Suitable compositions employed in the present invention can be applied as follows. During wear, at least about one drop, say, from about two to about five drops may be placed directly onto or near each lens whenever needed. Subsequently, the wearer rubs the eyelid over the worn lens for a sufficient time and at a sufficient pressure that enhances cleaning and disinfecting of the lens. Typically, such times are for at least about one second, say, from between about two seconds to about 10 seconds, e.g., at least about 5 seconds, typically, from about 5 to about 10 seconds, After rubbing, the procedure can be repeated one or more times, if desired. Preferably, the rubbing can be advantageously carried out using a circular motion, e.g., by gently tracing a fingertip over the lid as the eyelids are closed.

Typically, the cleaning and/or disinfecting process of the present invention can be carried out at least once weekly, preferably at least once daily, e.g., twice daily, or as often as necessary to effect comfortable wear of the lens and to clean and/or prophylactically clean lipid and/or protein deposits. By prophylactically clean is meant the method of the invention results in a reduction of bacteria on the interior and/or exterior surface of the lens. Alternately, such cleaning results in a reduction of bacteria on the lens surface ranging from about 20% to about 90% as measured before applying the solution and immediately after said rubbing by viable count method. In one embodiment of the invention, the wearer can blink at least once, preferably two or more times, between adding the drops and rubbing the eye. This improves dispersion of the drops along the lens surface.

The following specific experiments and examples demonstrate the method of the present invention and a solution employed in the method. However, it is to be understood that these examples are for illustrative purposes only and do not purport to be wholly definitive as to conditions and scope. All percentages are by weight of the solution, unless indicated otherwise. The examples presented are provided as a further guide to the practitioner of ordinary skill in the art and are not to be construed as limiting the invention in any way.

### EXAMPLE 1

This example discloses the preparation of a representative silicone hydrogel lens material used in the following Examples.

The formulation for the material is provided in Table 1 below.

TABLE 1

| Component | Parts by Weight |
|---|---|
| TRIS-VC | 55 |
| NVP | 30 |
| V₂D₂₅ | 15 |
| VINAL | 1 |
| n-nonanol | 15 |
| Darocur | 0.2 |
| tint agent | 0.05 |

The following materials are designated above: TRIS-VC=tris(trimethylsiloxy)silylpropyl vinyl carbamate, NVP=N-vinylpyrollidone, V₂ D₂₅ =a silicone-containing vinyl carbonate as previously described in U.S. Pat. No. 5,534,604, VINAL=N-vinyloxycarbonyl alanine, Darocur=Darocur-1173—a UV initiator, and tint agent=1,4-bis[4-(2-methacryloxyethyl)phenylamino]anthraquinone.

Silicone-hydrogel lenses made of the above formulation are cast molded from polypropylene molds. Under an inert nitrogen atmosphere, 45 mμl of the formulation are injected onto a clean polypropylene concave mold half and covered with the complementary polypropylene convex mold half. The mold halves are compressed at a pressure of 70 psi and the mixture is cured for about 15 minutes in the presence of UV light (6-11 mW/cm² as measured by a Spectronic UV meter). The mold is exposed to UV light for about 5 additional minutes.

The top mold half is removed and the lenses are maintained at 60° C. for 3 hours in a forced air oven to remove n-hexanol. Subsequently, the lens edges are ball buffed for 10 seconds at 2300 rpm with a force of 60 g. The lenses are then plasma treated as follows: The lenses are placed concave side up on an aluminum coated tray and the tray placed into a plasma treatment chamber. The atmosphere is produced by passing air at 400 sccm into the chamber through an 8% peroxide solution, resulting in an Air/H₂ O/H₂ O₂ gas mixture. The lenses are plasma treated for a period of 8 minutes (350 watts, 0.5 torr). The chamber is then backfilled to ambient pressure. The tray is then removed from the chamber, the lenses flipped over, and the procedure repeated to plasma treat the other side of the lenses. The plasma chamber is a direct current DC RFGD chamber manufactured by Branson GaSonics Division (Model 7104). This chamber is a cold equilibrium planar configuration that has a maximum power of 500 watts. Prior to any plasma treatment, the lenses in the chamber are prepumped to 0.01 torr to remove residual air in the chamber. Following plasma treatment, remaining processing includes extraction, hydration and autoclave sterilization. Extraction employs isopropanol at room temperature for 4 hours (during commercial manufacture a minimum of 48 hours following by extraction in water at about 85° C. for 4 hours is preferred). The lenses are then immersed in buffered saline for hydration. Autoclaving is carried out with the lenses, within vials, immersed in an aqueous packaging solution.

### Example 2

An aqueous solution (ReNu with MoistureLoc™) employed in the invention, useful for treating silicone hydrogel contact lenses worn in the eye, is prepared with the following ingredients in water as set out below in TABLES 2 and 3:

TABLE 2
pH 6.8-7.2 osmolality 270-300

| Ingredient | % w/w |
|---|---|
| Boric Acid | 0.8500 |
| Sodium Chloride | 0.1917 |
| Sodium Phosphate monobasic (NaPH₂O₄) | 0.1500 |
| Sodium Phosphate dibasic (Na₂PHO₄) | 0.3100 |
| HAP (30%) | 0.1000 |
| Tetronic 1107 | 1.0000 |
| Pluronic F-127 | 2.0000 |
| Polymer JR | 0.0200 |
| Alexidine 2HCl | 4.5000 ppm |

TABLE 3
pH 6.8-7.2 osmolality 240-280

| Ingredient | % w/w |
|---|---|
| Boric Acid | 0.8500 |
| Sodium Chloride | 0.0470 |
| Sodium Phosphate (monobasic) | 0.1500 |
| Sodium Phosphate (dibasic) | 0.3100 |
| HAP (30%) | 0.1000 |
| Tetronic 1107 | 1.5000 |
| Pluronic F-127 | 3.0000 |
| Polymer JR | 0.0200 |
| Pluronic P123 | 0.1000 |
| Alexidine 2HCl | 3.0000 ppm |

The formulation is prepared in bulk as follows: In a 316-grade stainless steel jacketed pressure kettle equipped with agitation, distilled water is added in the amount of about 50-55% of the total batch weight. The water is heated to 75 to 85° C. and with continued agitation the following batch quantities of the following ingredients are added, wherein after one ingredient is dissolved or hydrated, the next is added: sodium chloride, potassium chloride, boric acid, sodium borate, disodium edetate, PVP K-30, and Polymer JR 30M. The batch is heat sterilized at 121.1 to 123° C. for a minimum of 30 minutes. The total heat sterilization time has a desired target of 30-35 minutes, not to exceed 40 minutes. With continued agitation, the batch is cooled down with recirculating cold water through the outside jacket to a temperature not greater than 40° C. while maintaining positive internal pressure with sterile air. In a second phase of preparation, purified water is added to an appropriate clean mixing vessel to 35 to 40% of the total batch weight. With continued agitation, the following ingredients are added in the order listed: sodium phosphate, sorbic acid, the poloxamine, and propylene glycol, allowing each to dissolve or disperse before adding the next. This second solution is transferred to the first solution through a prefilter and sterilizing filter. The second-phase vessel and the filters are rinsed with a volume of purified water equivalent to 5 to 10% of the total batch weight. The batch is subjected to continued mixing while cooling the product to 20-35° C. for a minimum of 30 minutes. If necessary, the pH is adjusted to 7.0-7.40 at 25° C. with 2.5 N NaOH or 1N HCI. The finished solution should be aseptically passed through a sterile polishing filter prior to bottling.

### Example 3

20 μl of about 1×10⁵ cfu/ml of Pseudomonas aeruginosa mixed with 100% organic soil [a mixture of heat shocked saccharomyces cerevisiae and serum bovine albumin] are placed on one surface of individual silicone hydrogel PureVision® lenses prepared in accordance with Example 1, and left at room temperature for 10 minutes. The lenses were rinsed in Dulbecco's Phosphate Buffered Saline (DPBS) buffers based on Na₂HPO₄, NaH₂PO₄ and/or KH₂PO₄, in order to remove unbound bacteria and placed in 100 μl of the test solution of Example 2, or 10 ppm Alexidine Borate Buffer prepared in DPBS Lenses were picked up individually and gently rubbed for about 10 seconds and placed back in 100 μl of test solution for 5 minutes. The lenses were placed in a proper neutralization broth and the lenses were plated on TSA (Trypticase Soy Agar), growth medium for 48 hours. Viable colonies were counted and recorded as Colony Forming Units/lens (CFU/lens) to provide the number of attached microorganisms. Control lenses were treated similarly with Phosphate Buffered Saline (PBS). TABLES 4 and 5 utilize PureVision® silicone hydrogel lenses. The results are provided below:

TABLE 4
Staphylococcus aureus ATCC 6538 Inoculum 2.45 × 10⁵ cfu/ml

| | CFU's remaining |
|---|---|
| Test solution | on PureVision lens |
| PBS no rub | 175, 159 |
| PBS + rub | 91, 73 |
| ReNu ® with MoistureLoc ™ no rub | 131, 123 |
| ReNu ® with MoistureLoc ™ + rub | 57, 58 |
| BSD01 no rub | 153, 144 |
| BSD01 + rub | 81, 64 |
| 10 ppm Alexidine/PBS no rub | 58, 48 |
| 10 ppm Alexidine/PBS + rub | 35, 27 |

TABLE 5
Pseudomonas aeruginosa ATCC 9027 Incoculum 4.7 × 10⁵ cfu/ml

| | CFU's remaining |
|---|---|
| Test solution | on PureVision lens |
| PBS no rub | 271, 154 |
| PBS + rub | 115, 118 |
| ReNu ® with MoistureLoc ™ no rub | 239, 186 |
| ReNu ® with MoistureLoc ™ + rub | 48, 66 |
| BSD01 no rub | 263, 223 |
| BSD01 + rub | 71, 81 |
| 10 ppm Alexidine/PBS no rub | 182, 173 |
| 10 ppm Alexidine/PBS + rub | 59, 65 |

While there are described herein certain specific embodiments of the present invention, it will be manifest to those skilled in the art that various modifications may be made without departing from the spirit and scope of the underlying inventive concept and that the same is not limited to the particular forms herein described except insofar as indicated by the scope of the appended claims.

1. A method of treating a contact lens in the eye of a wearer comprising i) applying to said contact lens and/or eye an ophthalmically compatible contact lens solution comprising hypotonic to isotonic buffer, conditioning agent, sequestering agent, preservative, and/or biocidal agent; and ii) rubbing the eyelid associated with said eye over the contact lens for at least about one second.

2. The method of claim 1 wherein said ophthalmic solution further comprises at least one surfactant.

3. The method of claim 2 wherein said method further comprising the sequential steps of blinking and then rinsing the eye and the contact lens with an ophthalmic rinsing solution to dilute or substantially remove the contact lens cleaning solution from the eye.

4. The method of claim 1 wherein said contact lens is an extended wear lens worn within the range of about 7 days to about 30 days and said method improves resistance to lipid deposition.

5. The method of claim 1 wherein said solution is an aqueous solution comprising:
(a) 0.01 to 1.0 percent by weight of a cationic cellulosic polymer;
(b) at least one tonicity agent which is present in an amount of 0.01 to 10.0% by weight; and
(c) an effective amount of a buffering agent to maintain the pH from about 6 to about 8.

6. The method of claim 5, wherein said solution further comprises 0.1 to 5.0 percent by weight of one or more surfactants.

7. The method of claim 6, wherein at least one surfactant is a poly(oxyethylene)-poly(oxypropylene)-poly(oxyethylene) nonionic surfactant.

8. The method of claim 1, wherein the solution further comprises a wetting agent.

9. The method of claim 8, wherein the wetting agent is selected from the group consisting of glycerin, propylene glycol, mono or disaccharide, poly(ethylene glycol), ethoxylated glucose, and combinations thereof.

10. The method of claim 1, further comprising a polymeric nonionic demulcent.

11. The method of claim 1, wherein the solution further comprises an effective amount of a sequestering agent.

12. The method of claim 1, wherein the solution is applied by an eye-drop dispenser and is stored in a container capable of holding between about 1 and about 30 ml of the solution.

13. The method of claim 1, wherein the solution is employed to clean the lens.

14. The method of claim 13, wherein the solution is employed to clean and/or prophylactically clean lipid and/or protein deposits.

15. The method of claim 1 wherein said rubbing is carried out for at least about 5 seconds.

16. The method of claim 1 wherein said method results in a reduction of bacteria on the surface of said lens which contacts the eye ranging from about 20% to about 90%, as measured before applying the solution and immediately after said rubbing by viable count method.

17. The method of claim 16 wherein said method results in a reduction of bacteria on the surface of said lens which contacts the eye ranging from about about 40% to about 60% as measured before applying the solution and immediately after said rubbing by a viable count method.

18. The method of claim 1 wherein steps i) and ii) are repeated at least once.

19. The method of claim 1 wherein said rubbing is carried out by rubbing a fingertip over a closed eyelid.

20. The method of claim 1 wherein said preservative is selected from the group consisting of sorbic acid, poly(hexamethylene biguanide), polyquaternium 1, benzalkonium chloride, and chlorhexidine.

21. The method of claim 1 wherein said preservative comprises alexidine.

22. The method of claim 1 wherein said contact lens is a silicone hydrogel contact lens.

23. The method of claim 1 wherein said solution is a cleaning and/or hydrating solution.
